(19) United States
(12) Patent Application Publication
(10) Pub. No.: US 2026/0000728 A1
(43) Pub. Date: Jan. 1, 2026

(54) PEPTIDE HAVING ACE INHIBITORY ACTIVITY AND COMPOSITION CONTAINING THE SAME

(71) Applicant: Vedan International (Holdings) Limited, Grand Cayman (KY)

(72) Inventors: Kun Hsiang YANG, Taichung City (TW); Shin An YANG, Taichung City (TW); Chen Wen YANG, Taichung City (TW); Kuo Chiang HSU, Taichung City (TW)

(21) Appl. No.: 19/240,072

(22) Filed: Jun. 17, 2025

(30) Foreign Application Priority Data
Jun. 27, 2024 (TW) 113124109

(51) Int. Cl. A61K 38/06; A61P 9/12; C07K 5/083; C07K 5/097

(52) U.S. Cl. CPC A61K 38/06; A61P 9/12; C07K 5/0808; C07K 5/0821

(57) ABSTRACT

A peptide having angiotensin-converting enzyme (ACE) inhibitory activity and a composition containing the same are disclosed. The peptide is composed of three amino acids and can inhibit the activity of ACE. Therefore, administering a therapeutically effective amount of the peptide or a composition containing the peptide to an individual can effectively regulate the individual's blood pressure by bringing the blood pressure toward a normal value, thus producing the effect of treating or preventing hypertension or related diseases.

### FIELD OF THE INVENTION

The present invention relates to a small-molecule peptide and a use thereof. More particularly, the invention relates to a peptide having angiotensin-converting enzyme (ACE) inhibitory activity and a composition containing the peptide.

### DESCRIPTION OF THE RELATED ART

According to the information released by the World Health Organization, about one third of the world's population is subject to the health hazards of hypertension. Hypertension is a risk factor for stroke (a medical condition where a blood vessel in the brain bursts or is blocked), diabetes, dementia, kidney diseases, and cardiovascular diseases, and it can be known from the statistics of the Ministry of Health and Welfare of Taiwan that heart attack, cerebrovascular diseases, and hypertensive diseases, all of which are caused by hypertension, are among the top ten causes of death in Taiwan. Therefore, hypertension has become one of the most emphasized health issues in the world.

In addition to genetic factors, the onset of hypertension is related to one's living habits and eating habits. This explains why current clinical treatments of hypertension entail not only administering hypertension drugs, but also urging patients or high-risk groups for hypertension to improve their living habits. While hypertension drugs are effective in controlling blood pressure, long-term consumption may still result in side effects of the drugs, such as edema and kidney failure, and hypertension drugs are not suitable for patients with prehypertension. In consequence, comprehensive prevention of hypertension and related diseases is difficult to achieve.

### SUMMARY OF THE INVENTION

The primary objective of the present invention is to provide a peptide that has the activity to inhibit angiotensin-converting enzyme (ACE) and a composition that contains the peptide. The peptide can effectively regulate blood pressure by bringing it toward a normal value and thus has the effect of preventing and/or treating hypertension and related diseases.

To achieve the aforesaid effect, the peptide disclosed in the present invention as having ACE inhibitory activity has a sequence composed of three amino acids, and the sequence can be expressed as A1-A2-phenylalanine, wherein A1 is leucine, valine, or tyrosine while A2 is serine or threonine.

More specifically, the peptide having ACE inhibitory activity is LSF (Leu-Ser-Phe), VTF (Val-Thr-Phe), or YVF (Tyr-Val-Phe) and can bring blood pressure toward a normal value by inhibiting the activity of ACE in a living organism. Moreover, the peptide having ACE inhibitory activity can keep blood pressure within a normal range.

In one embodiment of this invention, the peptide having ACE inhibitory activity is used to prepare a composition for treating hypertension. Preferably, the active ingredients of the composition are LSF, VTF, LTF, and YVF, and the amount of the active ingredients (i.e., LSF, VTF, LTF, and YVF) in the peptide composition is 9.0-54.0 ng/mg.

Another embodiment of the invention discloses a method for treating hypertension or a hypertension-related disease in a subject in need thereof, comprising administering to the subject a therapeutically effective amount of a peptide having ACE inhibitory activity, wherein the peptide comprises an amino acid sequence selected from the group consisting of LSF (Leu-Ser-Phe), VTF (Val-Thr-Phe), and YVF (Tyr-Val-Phe).

Preferably, the peptide is administered orally.

Preferably, the peptide is administered at a dose ranging from 9.0 ng/mg to 54.0 ng/mg of the composition.

Preferably, the composition further comprises another peptide of LTF (Leu-Thr-Phe). For example, the composition comprises all four peptides: LSF, VTF, LTF, and YVF.

### BRIEF DESCRIPTION OF DRAWINGS

FIG. 1 is an LC-MS/MS chromatogram of YVF, VTF, LSF, and LTF.

FIG. 2 shows the difference in systolic blood pressure of each group of rats in each week with respect to the negative control group.

FIG. 3 shows the difference in diastolic blood pressure of each group of rats in each week with respect to the negative control group.

### DETAILED DESCRIPTION OF THE INVENTION

The present invention discloses a peptide having angiotensin-converting enzyme (ACE) inhibitory activity and a composition containing the same. More specifically, the peptide is composed of three amino acids, and the sequence of the peptide can be expressed as A1-A2-phenylalanine, with A1 being leucine, valine, or tyrosine, and A2 being serine or threonine. As the peptide disclosed herein can inhibit the activity of ACE, administering a therapeutically effective amount of the peptide or a composition containing the peptide to an individual can effectively regulate the individual's blood pressure by bringing the blood pressure toward a normal value, and in doing so, the intended effect of treating or preventing hypertension and related diseases can be achieved.

In one embodiment of the present invention, the amino acid sequence of the peptide is LSF (Leu-Ser-Phe), VTF (Val-Thr-Phe), LTF (Leu-Thr-Phe), or YVF (Tyr-Val-Phe). That is to say, a total of four peptides, or tripeptides, are disclosed (which are also collectively referred to as “the peptide disclosed herein” for the sake of simplicity). The molecular weights of the tripeptides are listed in Table 1.

TABLE 1
Molecular weights of the tripeptides
disclosed in the present invention

| Peptide | Molecular weight (Da) |
|---|---|
| LSF | 365.4 |
| LTF | 379.5 |
| VTF | 365.4 |
| YVF | 427.5 |

The peptide disclosed herein can be prepared by a chemical synthesis method, an artificial synthesis method, or a biological synthesis method. For example, solid-phase synthesis, which is currently one of the most frequently used synthesis methods, can be applied by: using a solid-phase support (e.g., a resin) as a foundation for the reactants; connecting the amino acids to the solid-phase support one after another to form the target peptide; and detaching the target peptide from the solid-phase support. If biological synthesis is used, a recombinant vector for expressing the target peptide will be constructed by recombinant technology and then transferred into a host cell for expression, and then the target peptide can be obtained using a purification and separation technique.

The peptide disclosed herein can also be obtained by separation from a plant protein hydrolysate. For example, rice protein is mixed with water at a ratio of 7.4% (w/v), before a hydrolase is added to effect hydrolysis and thereby produce a rice protein hydrolysate, wherein the hydrolase may be alcalase, bromelain, flavourzyme, or papain, with alcalase having the most preferred hydrolyzing effect, and wherein the hydrolysis conditions include a hydrolysis duration of at least 60-120 minutes and a pH value of 5.5-7.5. After that, the peptide disclosed herein (whose molecular weight is in the range from 360 to 430 Da) can be purified and separated from the rice protein hydrolysate.

The uppercase letters used in the sequence of the peptide disclosed herein are the abbreviations of the constituent amino acids and, where an explicit explanation is absent, shall be understood according to general common knowledge in the technical field to which the present invention pertains. For example, the sequence of the peptide disclosed herein is Leu-Ser-Phe when written as “LSF”, is Val-Thr-Phe when written as “VTF”, or is Leu-Ser-Phe when written as “LSF.”

The peptide disclosed herein has the activity to inhibit ACE and is therefore an ACE inhibitor (ACEI). The peptide can be used to prepare a peptide composition for treating and/or preventing hypertension and related diseases.

In one embodiment of the present invention, the peptide composition contains LSF, LTF, VTF, and YVF, and each milligram of the peptide composition includes a total of about 9.0-54.0 nanograms (ng) of LSF, LTF, VTF, and YVF.

The weight ratio among the LSF, LTF, VTF, and YVF in the peptide composition is 6:1.5-2:1:3.

The peptide composition may be added with other ingredients such as another peptide, a flavoring agent, and an excipient, depending on the use, dosage form, and recipients of the peptide composition, among other factors.

The composition may be a food, a complementary therapy product, or a drug.

The term “hypertension” refers to a condition in which an individual's blood pressure keeps rising and exceeds the value defined by medical convention as a high blood pressure. For example, according to the latest treatment guidelines released by the American Heart Association on Nov. 13, 2017 regarding the standard values of high blood pressure, hypertension is characterized by a systolic blood pressure at rest that is higher than 130 mmHg and a diastolic blood pressure at rest that is higher than 80 mmHg.

The term “prehypertension” refers to a condition in which the blood pressure values are between the normal blood pressure values and the values corresponding to hypertension. An individual diagnosed with prehypertension is generally viewed as a member of a high-risk group for hypertension.

The term “hypertension-related disease,” or simply “related disease,” refers to a disease for which high blood pressure is a risk factor, such as cardiovascular diseases, stroke, kidney diseases, dementia, and diabetes.

To demonstrate the technical features of the present invention and their effects, some experimental examples are described below with reference to the accompanying drawings.

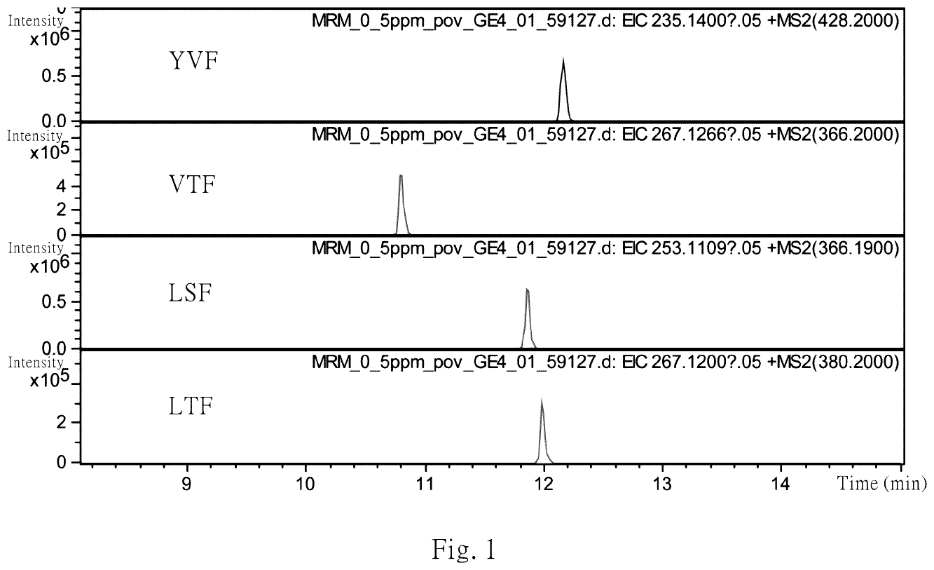

The peptides used in the following experimental examples were artificially synthesized or were separated from a plant protein hydrolysate and have been sequenced by liquid chromatography-tandem mass spectrometry (LC-MS/MS), with the sequencing results shown in FIG. 1.

The peptide composition used in the following experimental examples at least contained the following tripeptides as its active ingredients: LSF, LTF, VTF, and YVF; the peptide composition might contain other ingredients as well. The total amount of LSF, LTF, VTF, and YVF in each milligram of the peptide composition was about 9.0-54.0 nanograms (ng), and the weight ratio among LSF, LTF, VTF, and YVF is 6:1.5-2:1:3. For example, the weight ratio among the LSF, LTF, VTF, and YVF in the peptide composition is 54:14:9:27.

The animal tests in the following experimental examples complied with relevant ethical guidelines in animal testing.

The doses of the peptide composition in the following experimental examples were intended for the animal tests. The dose for use with a different animal can be calculated according to general common knowledge or applicable conversion standards in the technical field to which the present invention pertains.

### Example 1: Cell Test

The half maximal inhibitory concentration (IC₅₀) of each of the peptides LSF, LTF, VTF, and YVF in inhibiting ACE activity was determined by a method well known to a person of ordinary skill in the art, and the results are shown in Table 2.

TABLE 2
IC₅₀ of each tripeptide disclosed in the present
invention in inhibiting ACE activity

IC₅₀ in inhibiting ACE activity

| Peptide | mg/mL | μM |
|---|---|---|
| LSF | 0.44 | 1204 |
| LTF | 0.30 | 783 |
| VTF | 0.57 | 1552 |
| YVF | 0.82 | 1919 |

### Example 2: Animal Test (1)

Spontaneously hypertensive rats (SHR) were used as the subjects of the experiment, were each administered the peptide composition disclosed herein at a dose of 50 mg/kg body weight or 100 mg/kg body weight, and had their blood pressures measured at the time of administration (i.e., at the time point 0, or at 0 hour) and 2, 4, 6, and 8 hours after the administration in order to find out the variations in blood pressure, with the results shown in Table 3 and Table 4.

TABLE 3
Blood pressures and their variations when the dose
of the peptide composition was 50 mg/kg body weight

| Time point after feeding (hours) | 0 | 2 | 4 | 6 | 8 |
|---|---|---|---|---|---|
| Systolic blood pressure (SBP) | 166.5 | 160.6 | 157.0 | 151.2 | 150.4 |
| (mmHg) | | | | | |
| Systolic blood pressure variation | 0 | −5.9 | −9.5 | −15.3 | −16.1 |
| (pressure at specific time point − | | | | | |
| pressure at 0 hour) (mmHg) | | | | | |

TABLE 4
Blood pressures and their variations when the dose of
the peptide composition was 100 mg/kg body weight

| Time point after feeding (hours) | 0 | 2 | 4 | 6 | 8 |
|---|---|---|---|---|---|
| Systolic blood pressure (SBP) | 166.5 | 161.5 | 129.3 | 122.7 | 120.3 |
| (mmHg) | | | | | |
| Systolic blood pressure variation | 0 | −5.0 | −37.3 | −43.8 | −46.3 |
| (pressure at specific time point − | | | | | |
| pressure at 0 hour) (mmHg) | | | | | |

It can be known from the results in Table 3 that when the peptide composition was administered at the dose of 50 mg/kg body weight, the systolic blood pressures measured 4, 6, and 8 hours after the administration were reduced from that at 0 hour by 9.5, 15.3, and 16.1 mmHg respectively, and it can be known from the results in Table 4 that when the peptide composition was administered at the dose of 100 mg/kg body weight, the systolic blood pressures measured 4, 6, and 8 hours after the administration were reduced from that at 0 hour by 37.3, 43.8, 46.3 mmHg respectively, with all the magnitudes of reduction being greater than 20 mmHg. The results show that the peptide composition disclosed herein was effective in reducing blood pressure, and that the magnitude of reduction increased with dose and/or time.

### Example 3: Animal Test (2)

Six-week-old spontaneously hypertensive rats (SHR) were divided into 5 groups, each receiving the corresponding treatment stated below:
Negative control group (NC group): feeding with water
Positive control group (PC group): feeding with hypertension drug Captopril at 1 mg/kg
Low-dose group (L group): feeding with the peptide composition disclosed herein at a dose of 50 mg/kg
Medium-dose group (M group): feeding with the peptide composition disclosed herein at a dose of 100 mg/kg
High-dose group (H group): feeding with the peptide composition disclosed herein at a dose of 200 mg/kg

Besides, Wistar Kyoto rats were used as another control group (WKY group), which was treated by feeding with the peptide composition disclosed herein at a dose of 100 mg/kg.

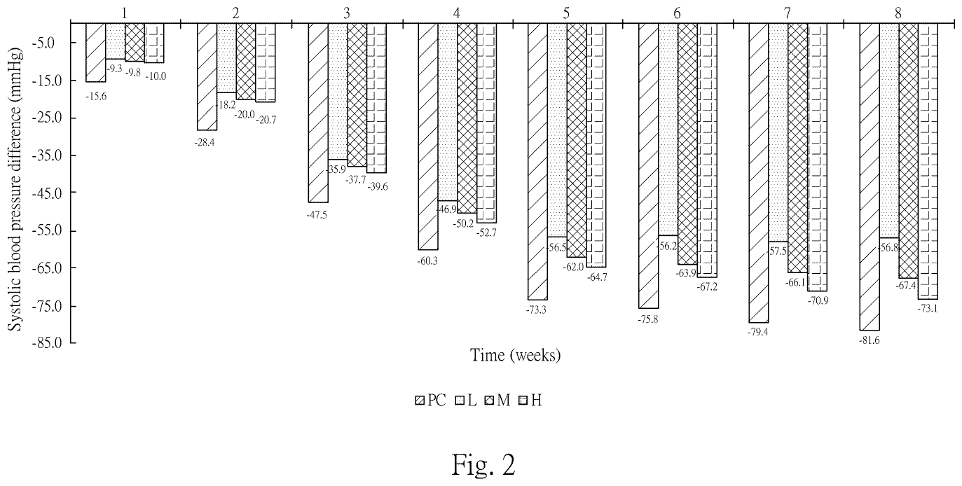

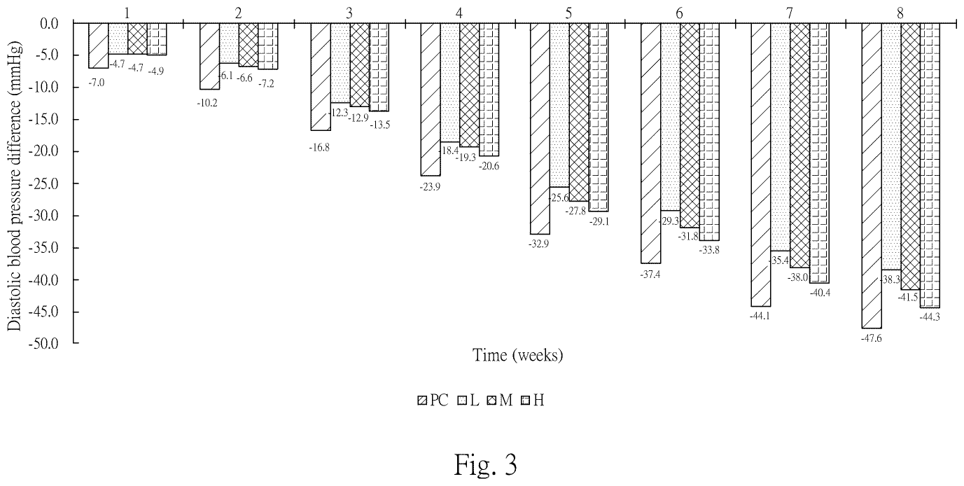

The experiment lasted for a total of 8 weeks. The diastolic and systolic blood pressures of the rats in each group were measured weekly, with the results shown in Table 5 and Table 6. The differences of each group from the negative control group were analyzed, and the results are shown in FIG. 2 and FIG. 3.

TABLE 5
Weekly systolic blood pressure of the rats in each group

| | Systolic blood pressure (mmHg) | | | | | | | | |
|---|---|---|---|---|---|---|---|---|---|
| Group | Week 0 | Week 1 | Week 2 | Week 3 | Week 4 | Week 5 | Week 6 | Week 7 | Week 8 |
| NC | 111.0 ± 9.3 | 125.8 ± 4.2 | 137.1 ± 6.7 | 156.1 ± 8.9 | 168.8 ± 6.4 | 181.4 ± 3.4 | 183.9 ± 4.9 | 187.4 ± 7.7 | 187.4 ± 7.7 |
| PC | 111.0 ± 7.2 | 110.2 ± 5.8 | 108.8 ± 4.2 | 108.6 ± 4.3 | 108.4 ± 3.9 | 108.1 ± 4.1 | 108.1 ± 2.0 | 108.0 ± 3.1 | 108.0 ± 2.5 |
| L | 112.1 ± 3.3 | 116.5 ± 6.4 | 118.9 ± 6.6 | 120.2 ± 8.5 | 121.9 ± 8.7 | 124.8 ± 8.7 | 127.7 ± 4.5 | 129.9 ± 6.6 | 132.8 ± 8.1 |
| M | 114.5 ± 4.6 | 116.0 ± 6.4 | 117.1 ± 6.2 | 118.3 ± 6.3 | 118.6 ± 5.7 | 119.3 ± 3.9 | 120.0 ± 7.4 | 121.3 ± 8.0 | 122.2 ± 6.1 |
| H | 114.5 ± 9.4 | 115.8 ± 5.3 | 116.5 ± 7.0 | 116.5 ± 5.0 | 116.0 ± 6.0 | 116.6 ± 6.0 | 116.7 ± 6.3 | 116.5 ± 4.8 | 116.5 ± 6.1 |
| WKY | 105.3 ± 3.7 | 105.0 ± 4.4 | 105.0 ± 3.4 | 105.2 ± 4.1 | 105.5 ± 4.5 | 105.9 ± 3.1 | 106.5 ± 3.1 | 105.9 ± 2.7 | 106.2 ± 1.6 |

TABLE 6
Weekly diastolic blood pressure of the rats in each group

| | Diastolic blood pressure (mmHg) | | | | | | | | |
|---|---|---|---|---|---|---|---|---|---|
| Group | Week 0 | Week 1 | Week 2 | Week 3 | Week 4 | Week 5 | Week 6 | Week 7 | Week 8 |
| NC | 82.0 ± 5.5 | 86.9 ± 6.5 | 89.2 ± 7.9 | 95.7 ± 8.0 | 102.8 ± 7.3 | 111.7 ± 7.0 | 116.2 ± 7.2 | 122.9 ± 8.4 | 126.5 ± 7.5 |
| PC | 80.2 ± 6.5 | 79.9 ± 6.3 | 79.0 ± 5.3 | 78.9 ± 6.4 | 78.9 ± 3.9 | 78.8 ± 3.8 | 78.8 ± 3.2 | 78.8 ± 5.0 | 78.8 ± 2.9 |
| L | 80.0 ± 8.9 | 82.2 ± 7.3 | 83.1 ± 9.0 | 83.4 ± 7.7 | 84.4 ± 4.9 | 86.1 ± 5.1 | 86.9 ± 3.7 | 87.5 ± 3.8 | 88.1 ± 2.8 |
| M | 81.0 ± 12.0 | 82.2 ± 7.3 | 82.5 ± 8.9 | 82.9 ± 4.6 | 83.5 ± 3.6 | 83.9 ± 6.3 | 84.4 ± 4.4 | 84.9 ± 4.3 | 85.0 ± 1.5 |
| H | 80.9 ± 12.0 | 82.0 ± 7.4 | 82.0 ± 5.2 | 82.2 ± 2.3 | 82.1 ± 2.5 | 82.6 ± 3.3 | 82.3 ± 4.4 | 82.5 ± 2.6 | 82.2 ± 4.1 |
| WKY | 68.1 ± 7.5 | 68.3 ± 6.6 | 68.2 ± 5.3 | 68.1 ± 3.9 | 68.2 ± 4.9 | 68.0 ± 3.7 | 68.2 ± 2.3 | 68.2 ± 3.2 | 68.2 ± 5.0 |

It can be known from the results in Table 5 and Table 6 that starting from week 1 of the experiment, the systolic blood pressures of the rats in the L, M, and H groups, which were administered the peptide composition disclosed herein, were significantly different from those of the PC group and the NC groups; that the difference between the week-3 systolic blood pressure of the rats in the L group and that of the NC group was greater than or equal to 20 mmHg; and that starting from week 2, the differences between the systolic blood pressures of the rats in the M, H, and PC groups and those of the NC group were greater than or equal to 20 mmHg. After the 8-week experiment, the systolic blood pressures of the rats in the L, M, and H groups, which were administered the peptide composition disclosed herein, were significantly different from those of the PC group and the NC group, with the differences between the systolic blood pressures of the rats in the L, M, and H groups and that of the NC group being 56.8, 67.4 and 73.1 mmHg respectively.

The foregoing results indicate that the peptide composition disclosed herein, whether administered at a high, medium, or low dose, caused a reduction in systolic blood pressure by more than 20 mmHg. According to the criteria disclosed in the assessment method for the auxiliary blood pressure regulation function of health foods, a substance under test is determined to have a blood pressure regulating effect if the difference caused by the substance under test between the systolic blood pressures before and after the test is great than or equal to 20 mmHg. Therefore, the peptide composition disclosed herein has been proved capable of blood pressure regulation and hence of producing the effect of treating or preventing hypertension and related diseases.

In addition, the blood pressures of the rats in the WKY group, which were fed with the peptide composition disclosed herein for 8 weeks in a row, did not show a significant difference, and this indicates that the peptide composition disclosed herein did not cause hypotension in rats with normal blood pressure.

What is claimed is:

1. A method for treating hypertension or a hypertension-related disease in a subject in need thereof, comprising administering to the subject a therapeutically effective amount of a peptide having ACE inhibitory activity, wherein the peptide comprises an amino acid sequence selected from the group consisting of LSF (Leu-Ser-Phe), VTF (Val-Thr-Phe), and YVF (Tyr-Val-Phe).

2. The method of claim 1, wherein the peptide is administered orally.

3. The method of claim 1, wherein the peptide is administered at a dose ranging from 9.0 ng/mg to 54.0 ng/mg of the composition.

4. The method of claim 1, wherein the composition further comprises another peptide of LTF (Leu-Thr-Phe).

5. The method of claim 4, wherein the composition comprises all four peptides: LSF, VTF, LTF, and YVF.

6. A peptide having ACE inhibitory activity, wherein the amino acid sequence of the peptide is LSF (Leu-Ser-Phe), VTF (Val-Thr-Phe), or YVF (Tyr-Val-Phe).
